(19) United States
(12) Patent Application Publication
(10) Pub. No.: US 2026/0108691 A1
(43) Pub. Date: Apr. 23, 2026

(54) METHOD FOR MANUFACTURING OROPHARYNGEAL MASK AND OROPHARYNGEAL MASK

(71) Applicant: TRANSIN BIOMEDICAL CO., LTD., Taoyuan City (TW)

(72) Inventors: CHIEH-MAO LIN, Taoyuan City (TW); TZU-HSUAN HUANG, Taoyuan City (TW); SHANG-YUAN WANG, Taoyuan City (TW)

(73) Assignee: TRANSIN BIOMEDICAL CO., LTD., Taoyuan City (TW)

(21) Appl. No.: 19/290,397

(22) Filed: Aug. 5, 2025

(30) Foreign Application Priority Data
Oct. 17, 2024 (TW) 113139589

(51) Int. Cl. A61M 16/04

(52) U.S. Cl. CPC A61M 16/047; A61M 2205/7536; A61M 2207/00; A61M 2210/1046

(57) ABSTRACT

The present application provides a method for manufacturing an oropharyngeal mask, including the following steps: ultrasonically laminating a first waterproof layer element, a body element, and a second waterproof layer element by an ultrasonic laminating device, to obtain an oropharyngeal mask. The body element includes a first bendable hanging portion, a hollow frame portion, and a second bendable hanging portion. A to-be-shielded opening is provided inside the hollow frame portion, and the first bendable hanging portion and the second bendable hanging portion are arranged on both sides of the hollow frame portion. Before the ultrasonic laminating device performs ultrasonic lamination, the second waterproof layer element is placed below the body element, the first waterproof layer element is placed above the body element, and the first waterproof layer element and the second waterproof layer element cover both sides of the to-be-shielded opening. Further, an oropharyngeal mask is also provided.

### CROSS-REFERENCE TO RELATED APPLICATION

This non-provisional application claims priority under 35 U.S.C. § 119(a) on Patent Application No(s). 113139589 filed in Taiwan, R.O.C. on Oct. 17, 2024, the entire contents of which are hereby incorporated by reference.

### BACKGROUND OF THE INVENTION

### Field of the Invention

The present disclosure relates to a method for manufacturing a mask and a mask product, and in particular to a method for manufacturing an oropharyngeal mask and an oropharyngeal mask.

### Description of the Related Art

In a process of oral treatment (or oral cleaning), if a person accidentally inhales fluids such as saliva or water, the person may feel uncomfortable due to choking and coughing. In practice, although a saliva ejector is used to remove oral fluids of the person, individuals who habitually breathe through their mouths, individuals who have undergone oral tumor surgery, or individuals with dementia may still accidentally inhale oral fluids, leading to choking and coughing. The individuals may then feel uncomfortable or the course of treatment needs to be interrupted immediately, and aspiration pneumonia may even be potentially caused.

Furthermore, in a process of oral treatment (e.g., tooth extraction), dental patients may accidentally swallow foreign objects such as extracted teeth or other dental instruments. The dental patients may feel uncomfortable or the course of treatment needs to be interrupted immediately, thereby causing emergencies and even threatening the safety of the dental patients.

In recent years, to avoid the foregoing situation, a dentist has placed a shield (i.e., an oropharyngeal mask) on the oropharynx of a patient (i.e., at the oropharyngeal airway in the patient's oral cavity) to prevent the patient from inadvertently inhaling fluids such as saliva or water or foreign objects such as extracted teeth or other dental instruments during oral treatment (or oral cleaning).

### BRIEF SUMMARY OF THE INVENTION

However, the design of conventional oropharyngeal masks primarily focuses on protectiveness for the oropharynx, without considering production efficiency of the oropharyngeal masks. In this case, the oropharyngeal masks cannot be mass-produced in a short time, resulting in high cost of the oropharyngeal masks. And then the oropharyngeal masks cannot be more widely applied to a process of oral treatment (or oral cleaning).

In view of this, how to resolve the foregoing problem about the conventional oropharyngeal masks and effectively improve the production efficiency of the oropharyngeal masks has become an urgent problem to be resolved in this technical field.

To resolve the foregoing problem, the present disclosure provides a method for manufacturing an oropharyngeal mask. The method includes the following steps: ultrasonically laminating a first waterproof layer element, a body element, and a second waterproof layer element by an ultrasonic laminating device, to obtain an oropharyngeal mask. The body element includes a first bendable hanging portion, a hollow frame portion, and a second bendable hanging portion. A to-be-shielded opening is provided inside the hollow frame portion, and the first bendable hanging portion and the second bendable hanging portion are respectively arranged on both sides of the hollow frame portion. Before the ultrasonic laminating device performs ultrasonic lamination, the second waterproof layer element is placed below the body element, the first waterproof layer element is placed above the body element, and the first waterproof layer element and the second waterproof layer element respectively cover both sides of the to-be-shielded opening of the hollow frame portion.

In some embodiments, when the ultrasonic laminating device performs ultrasonic lamination, the first waterproof layer element, the body element, and the second waterproof layer element are placed together on a protruding mold element, and the protruding mold element is located in the to-be-shielded opening of the hollow frame portion.

Further, the present disclosure also provides an oropharyngeal mask, including: a body element, including a first bendable hanging portion, a hollow frame portion, and a second bendable hanging portion, wherein a to-be-shielded opening is provided inside the hollow frame portion, and the first bendable hanging portion and the second bendable hanging portion are respectively arranged on both sides of the hollow frame portion; a first waterproof layer element, located above the body element and shielding the to-be-shielded opening; and a second waterproof layer element, located below the body element and shielding the to-be-shielded opening, wherein the first waterproof layer element, the body element, and the second waterproof layer element are ultrasonically laminated together by an ultrasonic laminating device.

In some embodiments, a first extension portion protrudes in a protrusion direction from a region, corresponding to the to-be-shielded opening, of the first waterproof layer element, and a second extension portion protrudes in the protrusion direction from a region, corresponding to the to-be-shielded opening, of the second waterproof layer element.

In some embodiments, the first waterproof layer element is a medical non-woven fabric.

In some embodiments, the second waterproof layer element is a high-fiber melt-blown non-woven fabric.

In some embodiments, at least one of the first bendable hanging portion, the hollow frame portion, and the second bendable hanging portion is a fuzzy yarn.

Accordingly, the technical means provided in the present disclosure can achieve advantageous effects unattainable by the prior art. Specifically, unlike conventional methods of manufacturing oropharyngeal masks through sewing, one of the advantageous effects achieved by the present disclosure is effective improvement in the production efficiency of the oropharyngeal masks. And then the oropharyngeal masks can be more widely applied to a process of oral treatment (or oral cleaning).

### BRIEF DESCRIPTION OF THE DRAWINGS

FIG. 1 is a flowchart for illustrating a method for manufacturing an oropharyngeal mask according to a first embodiment of the present disclosure.

FIG. 2A is a schematic diagram for illustrating a relative position among a first waterproof layer element, a body element, and a second waterproof layer element.

FIG. 2B is a schematic diagram for illustrating an oropharyngeal mask manufactured by using the method for manufacturing an oropharyngeal mask as shown in FIG. 1.

FIG. 2C is a schematic diagram for illustrating a structure of a body element.

FIG. 3 is a flowchart for illustrating a method for manufacturing an oropharyngeal mask according to a second embodiment of the present disclosure.

FIG. 4A is a schematic diagram for illustrating a relative position among a first waterproof layer element, a body element, a second waterproof layer element, and a protruding mold element.

FIG. 4B is a schematic diagram for illustrating an oropharyngeal mask manufactured by using the method for manufacturing an oropharyngeal mask as shown in FIG. 3.

FIG. 5 is a schematic diagram for illustrating a use state of an oropharyngeal mask.

### DETAILED DESCRIPTION OF THE INVENTION

The present disclosure will elaborate on the content through embodiments described below and the accompanying drawings to assist those skilled in the art in understanding objectives, features, and efficacy of the present disclosure.

It should be noted that various steps described in the present disclosure may be performed in order, in reverse order, or by appropriately altering or skipping the order during the control process. It should be noted that the description in the present disclosure that "the first step may be performed following the second step" may be interpreted as "directly performing the first step immediately after completing the second step" and/or "performing another step (e.g., a third step) after completing the second step before proceeding to the first step."

Furthermore, in the content described in the present disclosure, it should be noted that terms such as "first," "second," and "third" are used to distinguish between different elements and are not intended to limit the elements or indicate a specific order of the elements. It should be noted that in the content described below, the same elements or steps may be denoted by the same reference numerals.

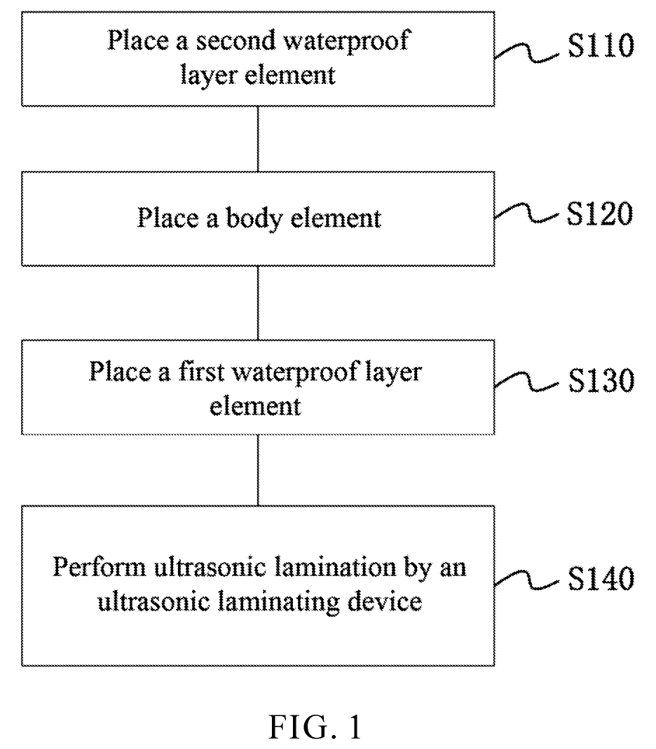

Referring to FIG. 1, FIG. 1 is a flowchart for illustrating a method for manufacturing an oropharyngeal mask according to a first embodiment of the present disclosure. In an example of FIG. 1, the method for manufacturing an oropharyngeal mask according to the present disclosure may include steps S110, S120, S130, and S140. The steps will be explained below in greater detail.

In some embodiments, in an example of FIG. 1, step S120 may be performed following step S110, step S130 may be performed following step S120, and step S140 may be performed following step S130.

In step S110, a second waterproof layer element is placed. More specifically, by performing step S110, the second waterproof layer element is placed in a specific region (e.g., a lamination region of an ultrasonic laminating device described later). In some embodiments, the second waterproof layer element may be a finished product known to those skilled in the art. In preferred embodiments, the second waterproof layer element may be a high-fiber melt-blown non-woven fabric, to improve the breathability and/or waterproof performance of the oropharyngeal mask by virtue of characteristics of the high-fiber melt-blown non-woven fabric, thereby improving the practicality of the oropharyngeal mask. In some embodiments, those skilled in the art may use an automated device (e.g., a robotic arm) to place the second waterproof layer element, thereby increasing the production capacity of the oropharyngeal mask by virtue of an operation of the automated device and manufacturing more oropharyngeal masks, so that the oropharyngeal mask can be more widely applied to a process of oral treatment (or oral cleaning).

In step S120, a body element is placed. More specifically, by performing step S120, the body element is placed above the second waterproof layer element. In some embodiments, those skilled in the art may use an automated device (e.g., a robotic arm) to place the body element, thereby increasing the production capacity of the oropharyngeal mask by virtue of an operation of the automated device and manufacturing more oropharyngeal masks, so that the oropharyngeal mask can be more widely applied to a process of oral treatment (or oral cleaning).

In some embodiments, the body element may include a first bendable hanging portion, a hollow frame portion, and a second bendable hanging portion. A to-be-shielded opening is provided inside the hollow frame portion, and the first bendable hanging portion and the second bendable hanging portion are respectively arranged on both sides of the hollow frame portion.

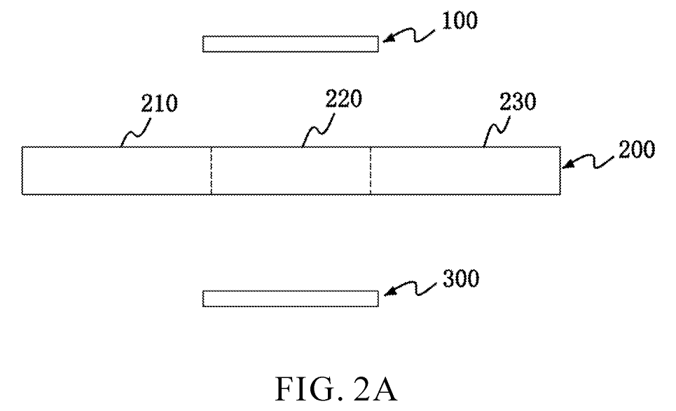

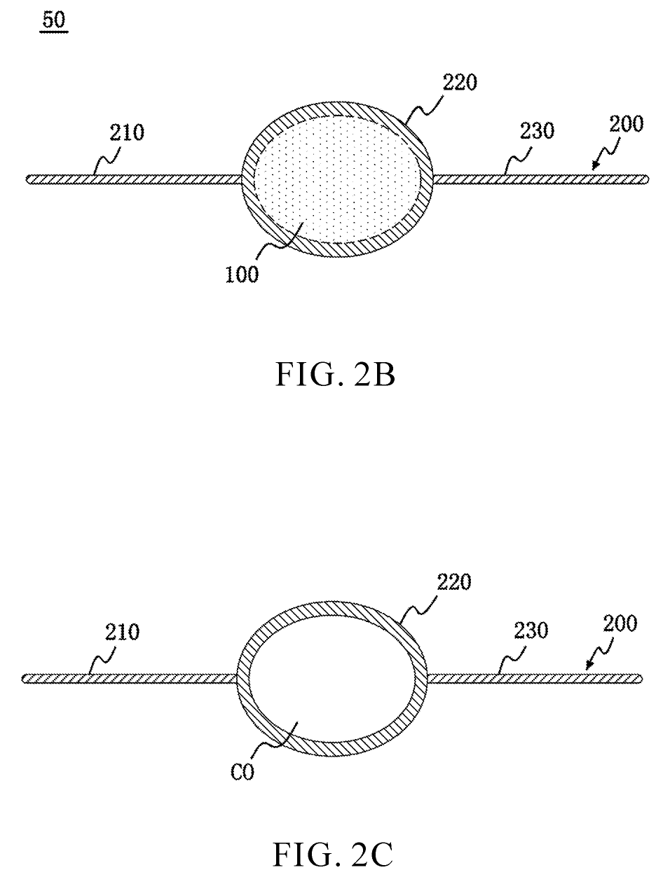

In an example of FIG. 2B described later, the body element 200 may include a first bendable hanging portion 210, a hollow frame portion 220, and a second bendable hanging portion 230. The first bendable hanging portion 210 is arranged on one side (e.g., a left side) of the hollow frame portion 220, and the second bendable hanging portion 230 is arranged on the other side (e.g., a right side) of the hollow frame portion 220. In some embodiments, those skilled in the art may adopt a symmetrical design to respectively arrange the first bendable hanging portion 210 and the second bendable hanging portion 230 on both sides (e.g., the left side and the right side opposite to the left side) of the hollow frame portion 220, thereby improving the use stability of the oropharyngeal mask (i.e., the stability of the oropharyngeal mask when worn in a dental patient's oral cavity to shield the oropharynx). Furthermore, in some embodiments, in an example of FIG. 2C described later, the hollow frame portion 220 may adopt a circular or elliptic ring structure, so as to form a to-be-shielded opening CO inside the hollow frame portion 220, thereby reducing an overall weight of the oropharyngeal mask and minimizing discomfort of the oropharyngeal mask to improve user experience. Also, the to-be-shielded opening CO is a connected region defined by the hollow frame portion 220, and the to-be-shielded opening CO may be shielded by waterproof layer elements (e.g., the first waterproof layer element and the second waterproof layer element) such as fabric, thereby preventing fluids such as saliva or water from accidentally flowing into the hollow frame portion 220, and improving protection for the oropharynx.

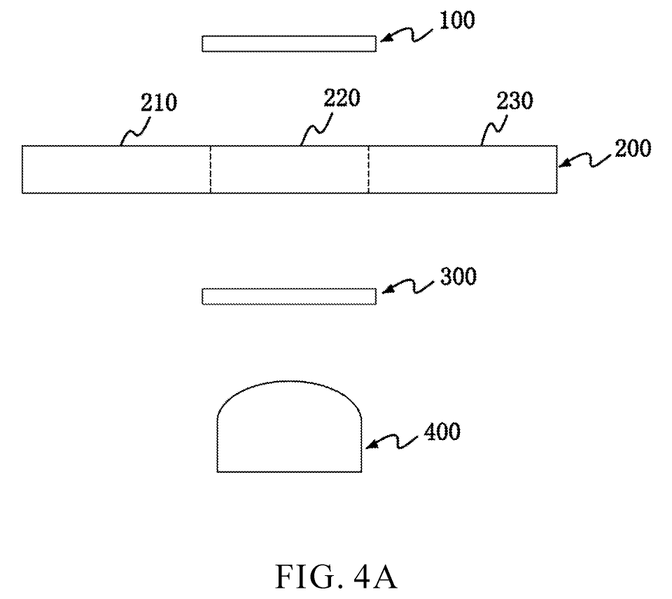

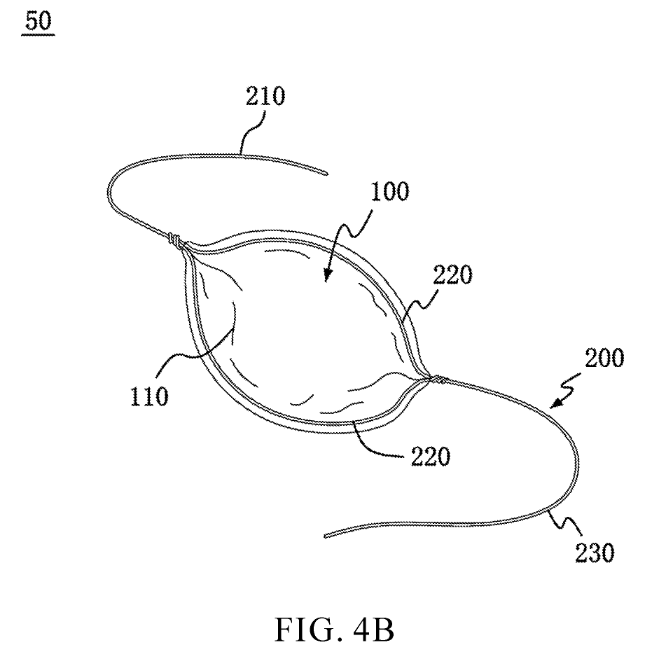

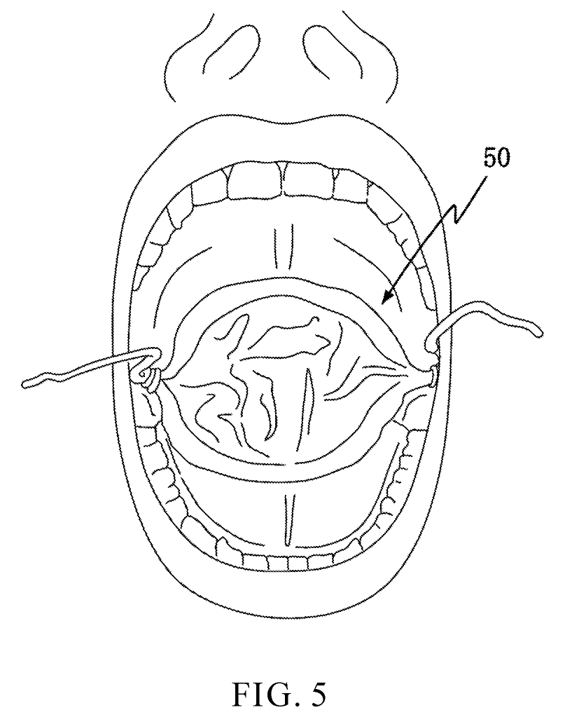

Also, in some embodiments, without limitation, the first bendable hanging portion 210 and the second bendable hanging portion 230 may be components made of a material bendable under force (e.g., a rigid plastic wire), and the hollow frame portion 220 may be a component made of a material having elasticity and being deformable under force (e.g., a rigid plastic wire). In preferred embodiments, at least one of the first bendable hanging portion 210, the hollow frame portion 220, and the second bendable hanging portion 230 may be, for example but not limited to, a fuzzy yarn. In an example of FIG. 4B described later, those skilled in the art may prepare a body element 200 of an oropharyngeal mask 50 by using a fuzzy yarn (e.g., by twisting two fuzzy yarns), to form a main structure of the oropharyngeal mask 50 (or referred to as a main frame of the oropharyngeal mask 50). Also, due to waterproof characteristics of the fuzzy yarn and suitability for contact with human skin, the configuration of the fuzzy yarn can improve the waterproof performance and wearing comfort of the oropharyngeal mask, thereby improving the practicality of the oropharyngeal mask and improving the user experience.

Additionally, in some embodiments, a size of the hollow frame portion 220 of the body element 200 depends on a size of the dental patient's oropharynx, so as to ensure coverage over the dental patient's oropharynx, thereby forming an oropharyngeal mask sufficient to shield the dental patient's oropharynx. More specifically, without limitation, the hollow frame portion 220 of the body element 200 may be configured as an elliptic ring structure having, for example, a major axis of 80 mm and a minor axis of 65 mm, and a to-be-shielded opening having, for example, a major axis of 70 mm and a minor axis of 55 mm is formed inside the hollow frame portion 220. Also, without limitation, the lengths of the first bendable hanging portion 210 and the second bendable hanging portion 230 may be separately configured into, for example, 90 mm.

In step S130, a first waterproof layer element is placed. More specifically, by performing step S130, the first waterproof layer element is placed above the body element. In some embodiments, the first waterproof layer element may be a finished product known to those skilled in the art. In preferred embodiments, the first waterproof layer element may be, for example, a medical non-woven fabric weighing 30 to 50 grams, to improve the breathability and/or waterproof performance of the oropharyngeal mask by virtue of characteristics of the medical non-woven fabric, thereby improving the practicality of the oropharyngeal mask. In some embodiments, those skilled in the art may use an automated device (e.g., a robotic arm) to place the first waterproof layer element, thereby increasing the production capacity of the oropharyngeal mask by virtue of an operation of the automated device and manufacturing more oropharyngeal masks, so that the oropharyngeal mask can be more widely applied to a process of oral treatment (or oral cleaning).

By performing steps S110 to S130, before various components of the oropharyngeal mask are ultrasonically laminated by the ultrasonic laminating device (i.e., step S140 is performed), relative positions and lamination relationships among the components of the oropharyngeal mask may be determined according to a specific placement order, so that a side structure of the oropharyngeal mask manufactured subsequently may sequentially include the first waterproof layer element, the body element, and the second waterproof layer element from top to bottom.

In an example of FIG. 2A described later, a second waterproof layer element 300 is placed below the body element 200, a first waterproof layer element 100 is placed above the body element 200, and the first waterproof layer element 100 and the second waterproof layer element 300 respectively cover both sides (i.e., an upper side and a lower side) of the to-be-shielded opening of the hollow frame portion 220 of the body element 200.

In step S140, an ultrasonic laminating device performs ultrasonic lamination. More specifically, by performing step S140, the ultrasonic laminating device ultrasonically laminates the placed first waterproof layer element, body element, and second waterproof layer element, to obtain an oropharyngeal mask. In some embodiments, the ultrasonic laminating device may be any model and/or specification of ultrasonic welding machine known to those skilled in the art. Unlike conventional methods of manufacturing oropharyngeal masks through sewing, the processing speed can be effectively increased by virtue of an operation of the ultrasonic laminating device, thereby effectively improving the production efficiency of the oropharyngeal masks. Consequently, more oropharyngeal masks can be manufactured, so that the oropharyngeal masks can be more widely applied to a process of oral treatment (or oral cleaning). In addition, the oropharyngeal masks manufactured by using conventional sewing methods are prone to yarn loosening, which may compromise protection of the oropharyngeal masks for the oropharynx. Therefore, lamination stability among the elements in the oropharyngeal mask can be effectively improved by virtue of an operation of ultrasonic lamination using the ultrasonic laminating device, and the compromise of protection of the oropharyngeal mask for the oropharynx is avoided, thereby improving the practicality of the oropharyngeal mask.

Referring to FIG. 2A, FIG. 2A is a schematic diagram for illustrating a relative position among a first waterproof layer element 100, a body element 200, and a second waterproof layer element 300. More specifically, before the ultrasonic laminating device performs an operation of ultrasonic lamination (i.e., step S140 is performed), the second waterproof layer element 300 is placed below the body element 200, and the first waterproof layer element 100 is placed above the body element 200, so as to manufacture an oropharyngeal mask having a side structure sequentially including the first waterproof layer element 100, the body element 200, and the second waterproof layer element 300 from top to bottom.

In some embodiments, the first waterproof layer element 100 and the second waterproof layer element 300 are respectively located above and below the body element 200, and respectively cover both sides (i.e., an upper side and a lower side) of the to-be-shielded opening of the hollow frame portion 220 of the body element 200, so as to respectively shield the upper side and the lower side of the to-be-shielded opening formed inside the hollow frame portion 220. Also, in some embodiments, the first waterproof layer element 100 and the second waterproof layer element 300 may be configured to fully wrap the body element 200.

Referring to FIG. 2B, FIG. 2B is a schematic diagram for illustrating an oropharyngeal mask 50 manufactured by using the method for manufacturing an oropharyngeal mask as shown in FIG. 1. In an example of FIG. 2B, the oropharyngeal mask 50 includes a first waterproof layer element 100, a body element 200, and a second waterproof layer element (not shown in FIG. 2B). The second waterproof layer element is located below the first waterproof layer element 100 and the body element 200.

Referring to FIG. 2C, FIG. 2C is a schematic diagram for illustrating a structure of a body element 200. In an example of FIG. 2C, the body element 200 may include a first bendable hanging portion 210, a hollow frame portion 220, and a second bendable hanging portion 230. The hollow frame portion 220 may adopt an elliptic ring structure, so as to form a to-be-shielded opening CO inside the hollow frame portion 220, thereby reducing an overall weight of the oropharyngeal mask and minimizing discomfort of the oropharyngeal mask to improve user experience. Also, the to-be-shielded opening CO is a connected region defined by the hollow frame portion 220, and the to-be-shielded opening CO may be shielded by waterproof layer elements (e.g., the first waterproof layer element and the second waterproof layer element) such as fabric, thereby preventing fluids such as saliva or water from accidentally flowing into the hollow frame portion 220, and improving protection for the oropharynx.

The first bendable hanging portion 210 and the second bendable hanging portion 230 of the body element 200 may be bent under force at appropriate positions, so that the oropharyngeal mask 50 can be worn on the dental patient's oral cavity after being appropriately adjusted according to mouth sizes and oral cavity depths of different dental patients, to protect the dental patient's oropharynx by virtue of the oropharyngeal mask 50.

The hollow frame portion 220 of the body element 200 may adopt an elliptic ring structure having, for example, a major axis of 80 mm and a minor axis of 65 mm, so as to form a to-be-shielded opening having, for example, a major axis of 70 mm and a minor axis of 55 mm inside the hollow frame portion 220, and the to-be-shielded opening may be shielded by the first waterproof layer element 100 and the second waterproof layer element, thereby preventing fluids such as saliva or water from accidentally flowing into the dental patient's oropharynx, to protect the dental patient's oropharynx by virtue of the oropharyngeal mask 50.

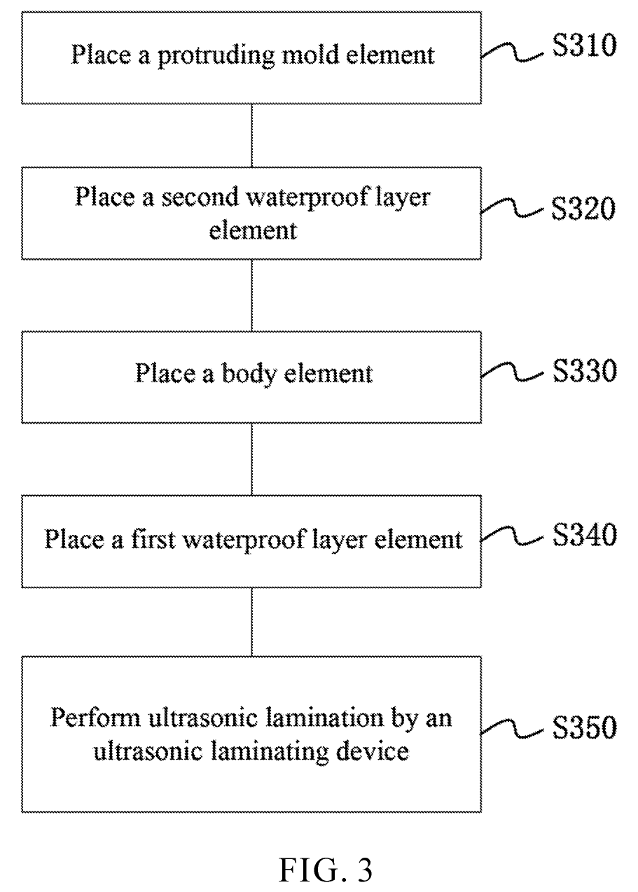

Referring to FIG. 3, FIG. 3 is a flowchart for illustrating a method for manufacturing an oropharyngeal mask according to a second embodiment of the present disclosure. In an example of FIG. 3, the method for manufacturing an oropharyngeal mask according to the present disclosure may include steps S310, S320, S330, S340, and S350. The steps will be explained below in greater detail.

In some embodiments, in an example of FIG. 3, step S320 may be performed following step S310, step S330 may be performed following step S320, step S340 may be performed following step S330, and step S350 may be performed following step S340.

In step S310, a protruding mold element is placed. More specifically, by performing step S310, the protruding mold element is placed in a specific region (e.g., a lamination region of an ultrasonic laminating device). In some embodiments, the protruding mold element may be fixedly placed in the lamination region of the ultrasonic laminating device, so as to continuously manufacture an oropharyngeal mask of FIG. 4B described later by virtue of the protruding mold element. In other words, after steps S310 to S350 are performed sequentially to manufacture a first oropharyngeal mask, step S320 may then be performed, so as to manufacture a second oropharyngeal mask by reusing the protruding mold element that was previously placed and fixed, thereby effectively improving the production efficiency of the oropharyngeal mask. In some embodiments, a cross-sectional size of the protruding mold element is smaller than a size of the hollow frame portion of the body element, so that the hollow frame portion of the body element may be sleeved on the protruding mold element, and the protruding mold element may be located in the to-be-shielded opening of the hollow frame portion of the body element.

In step S320, a second waterproof layer element is placed. More specifically, by performing step S320, the second waterproof layer element is placed above the protruding mold element.

In step S330, a body element is placed. More specifically, by performing step S330, the body element is placed above the second waterproof layer element, so as to sequentially form the body element, the second waterproof layer element, and the protruding mold element from top to bottom. Additionally, a center point of the hollow frame portion of the body element is configured to align with a center point of the protruding mold element, so that the hollow frame portion of the body element may be sleeved on the protruding mold element, and the protruding mold element may be located in the to-be-shielded opening of the hollow frame portion of the body element.

In step S340, a first waterproof layer element is placed. More specifically, by performing step S340, the first waterproof layer element is placed above the body element, so as to sequentially form the first waterproof layer element, the body element, the second waterproof layer element, and the protruding mold element from top to bottom.

In some embodiments, those skilled in the art may use an automated device (e.g., a robotic arm) to respectively place the protruding mold element, the second waterproof layer element, the body element, and the first waterproof layer element, thereby increasing the production capacity of the oropharyngeal mask by virtue of an operation of the automated device and manufacturing more oropharyngeal masks, so that the oropharyngeal mask can be more widely applied to a process of oral treatment (or oral cleaning).

In step S350, an ultrasonic laminating device performs ultrasonic lamination. More specifically, by performing step S350, the ultrasonic laminating device ultrasonically laminates the first waterproof layer element, the body element, and the second waterproof layer element placed on the protruding mold element, to obtain an oropharyngeal mask of FIG. 4B described later. In some embodiments, the ultrasonic laminating device may be any model and/or specification of ultrasonic welding machine known to those skilled in the art.

By placing the first waterproof layer element, the body element, and the second waterproof layer element above the protruding mold element, the ultrasonic laminating device performs ultrasonic lamination (i.e., step S350 is performed). Therefore, in a process of ultrasonic lamination, an oropharyngeal mask may be manufactured by using more materials of the first waterproof layer element and the second waterproof layer element, so that the first waterproof layer element and the second waterproof layer element of the manufactured oropharyngeal mask respectively have a first extension portion and a second extension portion (i.e., the first waterproof layer element and the second waterproof layer element have plasticity). Then, the hollow frame portion of the body element of the oropharyngeal mask can conform to the dental patient's oropharynx, and the size of the hollow frame portion of the body element can be finely adjusted, so that oropharyngeal masks of the same manufacturing specification can be more widely applied to different dental patients' oropharynx.

Referring to FIG. 4A, FIG. 4A is a schematic diagram for illustrating a relative position among a first waterproof layer element 100, a body element 200, a second waterproof layer element 300, and a protruding mold element 400. More specifically, before the ultrasonic laminating device performs ultrasonic lamination, the second waterproof layer element 300 is placed above the protruding mold element 400, the body element 200 is placed above the second waterproof layer element 300, and the first waterproof layer element 100 is placed above the body element 200, so as to manufacture, by virtue of the ultrasonic laminating device, an oropharyngeal mask having a side structure sequentially including the first waterproof layer element 100, the body element 200, and the second waterproof layer element 300 from top to bottom.

Referring to FIG. 4B, FIG. 4B is a schematic diagram for illustrating an oropharyngeal mask 50 manufactured by using the method for manufacturing an oropharyngeal mask as shown in FIG. 3. In an example of FIG. 4B, the body element 200 of the oropharyngeal mask 50 is made of a fuzzy yarn (for example, formed by twisting two fuzzy yarns). Also, a first extension portion 110 protrudes from a region, corresponding to the to-be-shielded opening of the hollow frame portion 220 of the body element 200, of the first waterproof layer element 100 of the oropharyngeal mask 50 in a protrusion direction (for example, a direction opposite to the position of the protruding mold element, i.e., an upward direction), and a second extension portion protrudes from a region, corresponding to the to-be-shielded opening of the hollow frame portion 220 of the body element 200, of the second waterproof layer element (not shown in FIG. 4B) of the oropharyngeal mask 50 in the protrusion direction (for example, the direction opposite to the position of the protruding mold element, i.e., the upward direction). Then, the first waterproof layer element 100 and the second waterproof layer element of the oropharyngeal mask 50 have plasticity, so as to finely adjust the size of the hollow frame portion 220 of the body element 200 in conformity to the dental patient's oropharynx (for example, an outwardly expanding force is applied moderately to increase the size of the hollow frame portion 220 of the body element 200), so that oropharyngeal masks of the same manufacturing specification can be more widely applied to different dental patients' oropharynx.

Referring to FIG. 5, FIG. 5 is a schematic diagram for illustrating a use state of an oropharyngeal mask 50. In an example of FIG. 5, in a process of oral treatment (or oral cleaning), a dentist may first hang the oropharyngeal mask 50 on a dental patient's oral cavity in an appropriate manner. Then the dental patient's oropharynx is protected by virtue of the oropharyngeal mask 50, and the dental patient is prevented from accidentally inhaling oral fluids, leading to choking and coughing, and/or is prevented from accidentally swallowing foreign objects such as extracted teeth or other dental instruments, leading to emergencies, thereby improving user experience and ensuring that oral treatment (or oral cleaning) can proceed smoothly.

The present disclosure has been further illustrated through the foregoing embodiments and the accompanying drawings. However, those skilled in the art may still make numerous modifications and variations without departing from the scope and spirit set forth in the claims of the present disclosure. Therefore, the scope of protection for the present disclosure should remain subject to the definitions provided in the claims and should not be limited by the embodiments disclosed in the specification.

What is claimed is:

1. A method for manufacturing an oropharyngeal mask, the method comprising the following steps:
ultrasonically laminating a first waterproof layer element, a body element, and a second waterproof layer element by an ultrasonic laminating device, to obtain an oropharyngeal mask,
wherein the body element comprises a first bendable hanging portion, a hollow frame portion, and a second bendable hanging portion, a to-be-shielded opening is provided inside the hollow frame portion, and the first bendable hanging portion and the second bendable hanging portion are respectively arranged on both sides of the hollow frame portion; and
wherein before the ultrasonic laminating device performs ultrasonic lamination, the second waterproof layer element is placed below the body element, the first waterproof layer element is placed above the body element, and the first waterproof layer element and the second waterproof layer element respectively cover both sides of the to-be-shielded opening of the hollow frame portion.

2. The method according to claim 1, wherein when the ultrasonic laminating device performs ultrasonic lamination, the first waterproof layer element, the body element, and the second waterproof layer element are placed together on a protruding mold element, and the protruding mold element is located in the to-be-shielded opening of the hollow frame portion.

3. The method according to claim 2, wherein the first waterproof layer element is a medical non-woven fabric.

4. The method according to claim 2, wherein the second waterproof layer element is a high-fiber melt-blown non-woven fabric.

5. The method according to claim 2, wherein at least one of the first bendable hanging portion, the hollow frame portion, and the second bendable hanging portion is a fuzzy yarn.

6. The method according to claim 1, wherein the first waterproof layer element is a medical non-woven fabric.

7. The method according to claim 1, wherein the second waterproof layer element is a high-fiber melt-blown non-woven fabric.

8. The method according to claim 1, wherein at least one of the first bendable hanging portion, the hollow frame portion, and the second bendable hanging portion is a fuzzy yarn.

9. An oropharyngeal mask, comprising:
a body element, comprising a first bendable hanging portion, a hollow frame portion, and a second bendable hanging portion, a to-be-shielded opening being provided inside the hollow frame portion, and the first bendable hanging portion and the second bendable hanging portion being respectively arranged on both sides of the hollow frame portion;
a first waterproof layer element, located above the body element and shielding the to-be-shielded opening; and
a second waterproof layer element, located below the body element and shielding the to-be-shielded opening,
wherein the first waterproof layer element, the body element, and the second waterproof layer element are ultrasonically laminated together by an ultrasonic laminating device.

10. The oropharyngeal mask according to claim 9, wherein a first extension portion protrudes in a protrusion direction from a region, corresponding to the to-be-shielded opening, of the first waterproof layer element, and a second extension portion protrudes in the protrusion direction from a region, corresponding to the to-be-shielded opening, of the second waterproof layer element.

11. The oropharyngeal mask according to claim 10, wherein the first waterproof layer element is a medical non-woven fabric.

12. The oropharyngeal mask according to claim 10, wherein the second waterproof layer element is a high-fiber melt-blown non-woven fabric.

13. The oropharyngeal mask according to claim 10, wherein at least one of the first bendable hanging portion, the hollow frame portion, and the second bendable hanging portion is a fuzzy yarn.

14. The oropharyngeal mask according to claim 9, wherein the first waterproof layer element is a medical non-woven fabric.

15. The oropharyngeal mask according to claim 9, wherein the second waterproof layer element is a high-fiber melt-blown non-woven fabric.

16. The oropharyngeal mask according to claim 9, wherein at least one of the first bendable hanging portion, the hollow frame portion, and the second bendable hanging portion is a fuzzy yarn.
